(19) United States
(12) Patent Application Publication
(10) Pub. No.: US 2002/0123524 A1
(43) Pub. Date: Sep. 5, 2002

(54) Treatment of vascular events using lipid-modifying compositions

(76) Inventors: Sander J. Robins, Southboro, MA (US); Hanna Bloomfield Rubins, Minneapolis, MN (US); Dorothea Collins, Guilford, CT (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 09/911,365

(22) Filed: Jul. 23, 2001

Related U.S. Application Data
(63) Continuation-in-part of application No. 09/189,699, filed on Nov. 10, 1998, now Pat. No. 6,264,960.

(51) Int. Cl. A61K 31/235

(52) U.S. Cl. 514/543

(57) ABSTRACT

The present invention pertains to methods for treating a patient who is at risk for a vascular (e.g., cardiovascular, cerebrovascular) event comprising administering to the patient an effective amount of a lipid-modifying drug. The present methods are particularly effective in patients having a lipid profile comprising a low Low Density Lipoprotein (LDL-C) and a low High Density Lipoprotein (HDL-C). Patients having low levels of LDL-C and HDL-C include diabetics. As a result, the present invention includes methods for treating diabetics by administering a lipid-modifying drug such as a fibric acid.

### RELATED APPLICATION

This application is a continuation-in-part of application Ser. No. 09/189,699, filed Nov. 10, 1998 entitled “Treatment of Vascular Events Using Lipid-Modifying Compositions” by Sander J. Robins, et al. The entire teachings of the above application are incorporated herein by reference.

### BACKGROUND OF THE INVENTION

Vascular disorders, including heart disease and stroke, persist as a leading cause of death in certain age and ethnic groups. Patients with diabetes are at a greater risk than other patient populations for these vascular disorders. A need exists, therefore, for improved treatments for patients at risk for vascular disorders. In particular, a need exists to reduce the occurrence and/or severity of vascular disorders in diabetic patients.

### SUMMARY OF THE INVENTION

The invention pertains to methods for treating a patient who is at risk for a vascular event (e.g., cardiovascular or cerebrovascular event) by administering to the patient an effective amount of a lipid or cholesterol modifying drug. In particular, the patient has a lipid profile with a low High Density Lipoprotein-Cholesterol (HDL-C) level and a low Low Density Lipoprotein-Cholesterol (LDL-C) level. A low HDL-C is less than about 40 mg/dL, and a low LDL-C is less than about 130 mg/dL. Administration of a lipid modifying drug such as a fibric acid increases HDL-C levels and reduces the occurrence of a vascular event.

A patient population that in particular significantly benefits from the methods of the present invention are diabetic individuals. Diabetics (e.g., individuals having diabetes mellitus) commonly have low HDL-C and low LDL-C levels. Diabetics are at greater risk for developing vascular disease than non-diabetics. Accordingly, the present invention relates to treating individuals who have diabetes and are at risk for vascular disease by administering a lipid or cholesterol modifying drug (e.g., a fibric acid). The present invention also embodies reducing the incidence or severity of a vascular disease, or preventing vascular disease in an individual who has diabetes by administering a lipid or cholesterol modifying drug.

The lipid/cholesterol modifying drugs include fibric acids or derivative thereof. Examples of fibric acids or derivatives thereof include gemfibrozil, fenofibrate, bezafibrate, ciprofibrate, clofibrate, clinofibrate and/or combinations thereof.

The present method embodies the treatment of various vascular events that can be characterized by a clot in a vessel (e.g., artery) or narrowing of the vessel. A clot which forms in a vessel can partially or fully block blood flow. Examples of such diseases are thrombotic disorders, myocardial infarction, angina, stroke, transient ischemic attack, thrombotic re-occlusion subsequent to a coronary intervention procedure and disorders in which at least one major coronary artery exhibits greater than about 50% stenosis. The treatment reduces the occurrence and/or severity of these vascular events.

The present invention results in at least about a 20% (e.g., 25%, 30%, 35%, 40%, 45% or 50%) reduction of the patient's triglyceride level, and/or at least about a 5% (e.g., 7% or 10%) increase in the level of HDL-C.

The present invention also embodies methods for preventing a cardiovascular and/or cerebrovascular event in an individual who has a lipid profile comprising a low HDL-C level and a low LDL-C level, by administering an effective amount of a fibric acid or derivative thereof in a carrier (e.g., a pharmaceutically acceptable carrier). The individual can also be at risk for a cardiovascular and/or cerebrovascular event.

Advantages of the present invention include the ability to effectively treat or prevent cardiovascular and/or cerebrovascular events in individuals who have low HDL-C and LDL-C levels. In particular, the present invention significantly reduces the occurrence of vascular disease in individuals having diabetes.

### DETAILED DESCRIPTION OF THE INVENTION

The present invention relates to methods for treating an individual (e.g., patient) who is at risk for a vascular event (e.g., cardiovascular and/or cerebrovascular event). The methods include administering an effective amount of a lipid/cholesterol-modifying drug. Individuals that benefit from these methods include those who have the following lipid profile: low High Density Lipoprotein-Cholesterol (HDL-C) and low Low Density Lipoprotein-Cholesterol (LDL-C) levels. Diabetics are a sub-population of patients who characteristically have low levels of HDL-C, and significantly benefit from the methods of the present invention. Administration of a lipid/cholesterol-modifying drug (e.g., fibric acid) results in an increase in the HDL-C level and a decrease in the occurrence or incidence of vascular disease. The methods of the present invention can also result in the prevention of vascular disease altogether.

High plasma LDL-C and low plasma HDL-C are recognized major CHD risk factors. There have been clinical trials that show that with drug therapy to lower a high LDL-C, the risk of a Coronary Heart Disease (CHD) event is significantly reduced. However, prior to the present invention and study described herein (See Examples 1 and 2), clinical trials did not demonstrate that therapy for increasing a low HDL-C level would significantly decrease the rate of vascular events, in particular, CHD and stroke events. The data described herein show that fibric acid, namely gemfibrozil (e.g., in a dose of 1200 mg/day), was used to increase low HDL-C levels. The study recruited men with known CHD (as established by a history of MI, etc), a low HDL-C (defined as less than or equal to about 40 mg/dL), and a low LDL-C (defined as less than or equal to about 140 mg/dL). Mean values of HDL-C and LDL-C at baseline were 32 mg/dL and 111 mg/dL, respectively. While gemfibrozil significantly increased HDL-C (compared to controls treated with placebo) and this increase in HDL-C was significantly related to a decrease in CHD and stroke events; gemfibrozil had no effect on levels of LDL-C. Thus, unlike clinical trials which have focused on a reduction of LDL-C, the data described herein for the first time demonstrated that without a reduction in LDL-C, CHD events and stroke could be reduced by an increase in HDL-C.

Assessment of HDL-C and LDL-C levels are generally associated with assessing the risk of cardiovascular and/or cerebrovascular disease. Traditionally, high levels of LDL-C are associated with coronary artery disease. However, the correlation between a high level of LDL-C and vascular events does not always occur. In fact, about 25% of the patients who are at risk for a cardiovascular and/or cerebrovascular event exhibit low HDL-C and low LDL-C levels. These patients have the same high risk of a recurrent vascular event or of a new vascular event, as other patients with low HDL-C who may not also have a low LDL-C. This population was thus targeted in the data described herein. Subjects with low HDL-C and low LDL-C levels, and known vascular disease (documented by a history of past MI, coronary revascularization procedure, angina with ischemic changes, or a positive coronary angiogram showing greater than 50% stenosis of at least one major coronary artery) were studied. These patients also exhibited a lipid profile characterized by a low HDL-C (about less than 40mg/dL) and a low LDL-C (about less than 140 mg/dL) based on the entrance lipid criteria for the population enrolled in the studied, as well as the mean baseline for the population. Prior to the present invention, physicians generally did not associate individuals having low levels of both HDL-C and LDL-C with a greater risk for vascular disease. Consequently, prior to the present invention, a physician would not have generally placed an individual having this lipid profile on any specific drug regimen for preventing vascular disease. On the contrary, the present invention demonstrates that administering a fibric acid to an individual having low levels of HDL-C and LDL-C reduces the risk of vascular disease, and therefore provides a new treatment plan for this patient population.

Administering a lipid modifying drug (e.g., fibric acid or derivative thereof) in an effective amount results in a reduction of recurrent cardiovascular and/or cerebrovascular events. Use of the methods described herein results in a reduction by at least about 10% (e.g., 15%, 20%, 25%) in the number of recurrent heart attacks, cardiac deaths and/or strokes. The data in Example 1 shows that administration of a fibric acid (e.g., gemfibrozil) reduced the number of heart attacks, cardiac deaths and stroke by a surprising 24%.

Administering a lipid modifying drug in a patient with low HDL-C and low LDL-C levels also results in an increase of the HDL-C level and/or reduction of the triglyceride level in a patient. In particular, use of the methods described herein results in an increase of at least about 5% (e.g., 7% 10%, 15%, or 20%) of HDL-C level, and/or a reduction in the triglyceride level by at least about 20% (e.g., 25%, 30%, 35%, 40%, 45% or 50%).

The methods of the present invention also relate to methods for reducing the occurrence (e.g., incidence) or severity of a cardiovascular and/or cerebrovascular event in a patient who is at risk for such a disorder. Reducing the occurrence or incidence of a cardiovascular and/or cerebrovascular disorder refers to reducing the probability that a patient will develop a cardiovascular and/or cerebrovascular disorder, or delaying the onset of the disorder. Reducing the severity of a cardiovascular and/or cerebrovascular disorder refers to a reduction in the degree of at least one symptom of the disorder.

The present invention also embodies methods for preventing the onset of a cardiovascular and/or cerebrovascular disorder in an individual having a low HDL-C and low LDL-C, by administering a lipid modifying drug. The individual can be at risk for developing a cardiovascular and/or cerebrovascular disorder, as demonstrated by exhibiting at least one symptom of a cardiovascular and/or cerebrovascular disorder or having a history of a cardiovascular and/or cerebrovascular disorder, as described herein. Preventing the onset of a cardiovascular or cerebrovascular event refers to delaying the onset of the event or death; or preventing the event altogether.

In particular, the present invention involves treating patients who are at risk for a cardiovascular and/or cerebrovascular event and have a low HDL-C and low LDL-C lipid profile. A low HDL-C level is a level less than about 40 mg/dL, and a low LDL-C level is a level less than about 130 mg/dL. Lipoproteins are complexes which contain both a lipid and protein. Most of the lipids in plasma are present as lipoproteins and are transported as such. Lipoproteins are characterized by their flotation constants (e.g., densities). Various classes of lipoproteins exist and include HDLs and LDLs. LDLs are particularly rich in cholesterol esters. The methods also include patients who have a triglyceride level of less than about 320 mg/dL (e.g., less than about 310 or 300 mg/dL).

Lipoproteins levels and triglyceride levels are measured and assessed using routine methods known in the art. Commercially available kits and assays may be used to evaluate the level of HDL-C, LDL-C and level of triglyceride.

In another embodiment, the present invention relates to treating diabetics (e.g., individuals having Type 2 diabetes) who are at risk for vascular disease. Two types of diabetes mellitus exist: 1) Type 1 diabetes (also referred to as “juvenile diabetes”), and 2) Type 2 diabetes (also referred to as “adult-onset diabetes”). Individuals with Type 2 diabetes, the most common form of diabetes, characteristically have low levels of HDL-C (e.g., less than about 40 mg/dL). Type 2 diabetics are at higher risk for a vascular event than Type 1, and are 2-4 times likely to experience a vascular event than non-diabetics. The data described in Example 2 herein show that administration of a fibric acid, a lipid modifying drug, significantly reduces the incidence or occurrence of a vascular event in diabetic individuals (individuals with Type 2 diabetes). In fact, the data described in Example 2 show that treatment of a diabetic with a fibric acid reduces the risk of experiencing a vascular event by about 30%.

Type 2 diabetes is a disease which involves abnormally high or unhealthy blood glucose (e.g., sugar) levels. Often, the individual's inability to make sufficient amounts of insulin, or properly functioning insulin cause these abnormal blood glucose levels. Accordingly, the present invention relates to reducing the risk or incidence of vascular disease in Type 2 diabetics.

The present invention also embodies reducing the risk or incidence of vascular disease in individuals in a “pre-diabetic state.” This pre-diabetic state is referred to as hyperinsulinemia or insulin resistance. Hyperinsulinemia is a condition in which non-functioning insulin or improperly functioning insulin is circulating in the blood. This insulin cannot properly mobilize the glucose and get it into the cells, resulting in high levels of this improperly functioning insulin in the blood stream. After prolonged periods of time during which the pancreas makes this improperly functioning insulin, the pancreas may stop making insulin. As a result, at least 3 classes of patients exist: 1) patients with Type 2 diabetes and hyperinsulinemia, 2) patients with Type 2 diabetes and no hyperinsulinemia, and 3) patients having hyperinsulinemia, but no Type 2 diabetes. All 3 classes of individuals are encompassed by the present invention. Table 4 of Example 2 shows that treatment with a fibric acid in diabetics (which can include patients with hyperinsulinemia), individuals with hyperinsulinemia (which can include Type 2 diabetics), and individuals with hyperinsulinemia alone (which excludes Type 2 diabetics) results in a reduction of vascular events by 30.3%, 24,2% and 21.7%, respectively.

Determining whether an individual has Type 2 diabetes and/or hyperinsulinemia can be done using assays that are well known in the art. Several assays are commercially available. The levels that define whether an individual has Type 2 diabetes or hyperinsulinemia vary depending on the type of assay used. An example of an assay that can measure glucose tolerance or glucose levels is one that uses glucose oxidase as a reagent. Using such a test, levels of fasting glucose of greater than or equal to about 126 mg/dL characterize a type 2 diabetic. An assay referred to as “Coat-A-Count” (Diagnostic Products, Los Angeles, Calif.) is an example of an assay that measures insulin. Levels of fasting insulin above about 40 μU/mL using the Coat-A-Count assay are levels that characterize an individual with hyperinsulinemia. For the data described in Example 2, insulin resistance (hyperinsulinemia) was defined by the levels of insulin determined in the Framingham Offspring Population. Meigs J B, et al., Metabolic Risk Factors Worsen Continuously Across the Spectrum of Nondiabetic Glucose Tolerance, The Framingham Offspring Study. Ann Intern Med 128:524-533 (1998). Subjects, described in Example 2, were designated as diabetic by having a history of diabetes or a fasting plasma glucose of about 126 mg/dL or greater and as being hyperinsulinemic with a fasting plasma insulin of about 40 μU/mL or greater. This value of insulin marked the 90th percentile of values for subjects with normal glucose tolerance in the Framingham Offspring Study. This value also corresponded to the level of insulin at which there was a significant increase in CHD events in Example 2.

The data described herein show that treating a diabetic with a lipid-modifying drug such as a fibric acid results in a decrease in cardiovascular or cerebrovascular events. Diabetics treated with fibric acid had less heart attacks (CHD, angina, MIs), less strokes, and experienced less death. Not only did treatment with a fibric acid result in less CHD deaths (e.g., deaths followed by or as the result of CHD), but also, surprisingly, reduced the incidence of death not related to CHD (referred to as “all-cause mortality”). CHD refers to cardiovascular events, described herein, and include, for example, MI and angina. In Example 2, diabetics taking gemfibrozil experienced 30.3% reduction in the risk of a cardiovascular or cerebrovascular event. See Table 4. Hence, the methods of the present invention embodies reducing the risk or incidence of cardiovascular events and cerebrovascular events in diabetics by at least about 20% (e.g., 25%, 30%, 35%, 40%, 45%, or 50%).

In one instance, the risk of death related to or as the result of CHD was reduced by about 41%, and all cause mortality was reduced by about 25%. Thus, the present invention includes reducing the incidence of death related to cardiovascular or cerebrovascular events by at least about 30% (e.g., 35%, 40%, 45%, or 50%), and the incidence of death unrelated to cardiovascular or cerebrovascular events by at least about 15% (e.g., 20%, 25%, 30%, or 35%). Treatment of diabetics with a fibric acid results in increase in HDL-C levels and decreases in triglyceride levels, as further described herein.

As defined herein, a vascular event (e.g., vascular disease) refers to a disease or disorder of a blood vessel and/or relating to the circulation of, for example, the heart (e.g., cardiovascular event) or brain (e.g., cerebrovascular event). A cardiovascular and/or cerebrovascular event can be a thrombotic disorder. A thrombotic disorder/event occurs, for example, when a clot forms and lodges within a blood vessel. The blockage may fully block or partially block the blood vessel causing a thrombotic disorder. Other examples of cardiovascular and/or cerebrovascular events include a narrowing or constriction of a blood vessel resulting in myocardial infarction (MI), angina, stroke, transient ischemic attack (TIA); thrombotic re-occlusion subsequent to a coronary intervention procedure; or disorders in which at least one major coronary artery exhibits greater than 50% stenosis. Two phases of a cardiovascular and/or cerebrovascular event can exist, an ischemic stage and a necrotic stage. A patient may suffer from ischemia in which a decrease of blood flow may occur. This decrease in blood flow causes a decrease in tissue oxygenation. After prolonged ischemia, the tissue may undergo necrosis which is death of the tissue. Patients who are at risk for a cardiovascular and/or cerebrovascular event can exhibit elevated levels of ischemic markers and/or necrosis markers. Hence, a way of assessing the presence, absence, or extent of a cardiovascular and/or cerebrovascular event is to assess these markers, as well as other markers now known or later developed.

Patients who are at risk for a cardiovascular and/or cerebrovascular event are patients who manifest at least one symptom indicative of a vascular disorder/event. Symptoms that are indicative of a coronary-related vascular event, for example, include chest pain, abnormal electrocardiograms, elevated levels of ischemic markers, necrosis markers, or thrombin/fibrin generation markers. Such markers include, but are not limited to, Creatine Kinase with Muscle and/or Brain subunits (CKMB), D-Dimer, F1.2, thrombin anti-thrombin (TAT), soluble fibrin monomer (SFM), fibrin peptide A (FPA), myoglobin, thrombin precursor protein (TPP), platelet monocyte aggregate (PMA) and troponin. Patients who are at risk also include patients having a history of a thrombotic event (e.g. disorder), including Coronary Heart Disease (CHD), stroke, or TIAs. A history of CHD can include, for example, a history of MI, coronary revascularization procedure, angina with ischemic changes, or a positive coronary angiogram (e.g., showing greater than about 50% stenosis of at least one major coronary artery).

Lipid Modifying Drugs, Fibric Acid and Derivatives Thereof

A lipid modifying drug refers to a drug (e.g., composition) that can influence the lipoprotein levels. Here, a lipid modifying drug refers to a drug which can increase the level of HDL-C and/or reduce the triglyceride level. The present invention further relates to administering drugs that are now known, and developed in the future. Many of these drugs are known and commercially available. Well recognized lipid modifying drugs are fibric acid or fibric acid derivatives. Examples of such compositions are gemfibrozil, fenofibrate, bezafibrate, ciprofibrate, clofibrate, and clinofibrate. These drugs are commercially available, and can also be made using methods known in the art. A lipid-modifying drug and its dosages and modes of administration are described in U.S. Patent No. 4,788,183.

Gemfibrozil refers to 5-(2,5-dimethylphenoxy)-2,2-dimethylpentanoic acid, and its pharmaceutically acceptable salts and esters. Gemfibrozil is commercially available under the trademark LOPID® from Parke, Davis & Company, Detroit Mich., and can be prepared by the methods disclosed in U.S. Pat. No. 3,674,836. There are several reported syntheses of gemfibrozil and certain analogues. For example, U.S. Pat. No. 3,674,836 (1972) discloses gemfibrozil and its analogues thereof, as well as a process for preparing them. U.S. Pat. No. 3,707,566 (1987) discloses a process for preparing gemfibrozil, and methods of making cholesterol modifying compounds are described in U.S. Patent No. 5,530,145.

Administration and Dosages

The terms “pharmaceutically acceptable carrier” or a “carrier” refer to any generally acceptable excipient or drug delivery device that is relatively inert and non-toxic. A preferred embodiment is to administer the lipid modifying drug orally, (e.g., tablet or capsule form). Exemplary carriers include calcium carbonate, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17th Ed., Mack Pub. Co., Easton, Pa.).

Suitable carriers (e.g., pharmaceutical carriers) also include, but are not limited to sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. Such preparations can be sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. They can also be combined where desired with other active substances, e.g., enzyme inhibitors, to reduce metabolic degradation. A carrier (e.g., a pharmaceutically acceptable carrier) is preferred, but not necessary to administer a lipid modifying drug.

The lipid-modifying (e.g., lipid modulating) compositions of the present invention can be administered orally. The composition can be administered in a single dose or in more than one dose over a period of time to confer the desired effect.

One or more lipid modifying drugs that include fibric acids and/or fibric acids in combination with another class of lipid drugs, such as niacin or statins, can be administered. When administering more than one lipid modifying drugs, the administration of the drugs can occur simultaneously or sequentially in time. The antagonists can be administered before and after one another, or at the same time. Thus, the term “co-administration” is used herein to mean that the drugs will be administered at times to increase HDL-C, and/or reduce the occurrence, severity or the vascular disease. The methods of the present invention are not limited to the sequence in which the various drugs are administered, so long as the drugs are administered close enough in time to produce the desired effect. The methods also include co-administration with other drugs that used to treat vascular diseases, diabetes or other diseases described herein.

The actual effective amounts of lipid modifying drug can vary according to the specific drug being utilized, the particular composition formulated, the mode of administration and the age, weight and condition of the patient, for example. As used herein, an effective amount of a lipid-modulating drug is an amount of the drug which is capable of increasing the level of HDL-C and/or reducing the triglyceride level. Dosages for a particular patient can be determined by one of ordinary skill in the art using conventional considerations, (e.g. by means of an appropriate, conventional pharmacological protocol). As described herein, a daily oral dose for a patient of gemfibrozil, for example, is about 800 -1300 mg/day, and preferably between 900-1200 mg/day.

A description of preferred embodiments of the invention follows.

### Examplification

### EXAMPLE 1

Fibric acid decreases the incidence of vascular events in patients having low HCL and Low LDL-C

This study, the Veterans Administration HDL-C Intervention Trial (VA-HIT), was a seven year, 20 center, randomized, placebo-controlled, double blind secondary prevention trial designed to determine if a fibric acid (e.g., gemfibrozil) reduced the incidence of myocardial infarction (MI) and CHD death in male patients who have low levels of both HDL-C and LDL-C. Power calculations indicated that a sample size of 2500 would yield 90% power to detect a 20% difference between the treatment groups for the primary endpoint (time to MI or CHD death) using a two-tailed alpha of 0.05 in an intention to treat analysis assuming a cumulative event rate in the placebo group of 30%. Primary endpoints and the secondary endpoint, stroke, were adjudicated by blinded committees using standard criteria. Gemfibrozil (1200mg/day, orally) was selected with the goal of raising HDL-C and lowering triglycerides (TG) without affecting LDL-C.

The study included men with known coronary heart disease (CHD) as documented by a history of past myocardial infarction (MI, coronary revascularization procedure, angina with ischemic changes, or a positive coronary angiogram (showing >50% stenosis of at least one major coronary artery) and a fasting plasma (or serum) lipid profile characterized by a low high-density lipoprotein (HDL)—cholesterol as well as a low Low-Density lipoprotein (LDL) -cholesterol. With gemfibrozil treatment (1200 mg/day) there was a significant reduction of new or recurrent CHD events, including nonfatal MI, CHD death, as well as certain forms of stroke or TIA.

“Low HDL-C” was defined as less than 40 mg/dL and “low LDL-C” was defined as less than 140 mg/dL values which are based on the entrance lipid criteria for the population enrolled in VA-HIT. With these lipid criteria, the mean entrance value at baseline of HDL-C was 32 mg/dL and of LDL-C was 111 mg/dL.

Other fibric acid derivatives that are structurally similar to gemfibrozil including but not limited to 1) fenofibrate, 2) bezafibrate, and 3) ciprofibrate have a similar significant clinical benefit as gemfibrozil when used to treat patients belonging to this subpopulation.

The benefits observed in this study were seen in following subgroups:

1) Patients who were young as well as old (>65 years)

2) diabetics as well as non-diabetics

3) patients with lower values of triglycerides (<161 mg/dL) as well as higher (from 161 to 300 mg/dL) values.

A clinical benefit has been shown in CHD patients who do not have a high (total) cholesterol or high LDL-C by treating them with a particular lipid-modifying drug.

2531 patients were enrolled and followed for a median of 5 years. Compliance, lipid changes, endpoints rates, losses to follow-up, and crossovers to active treatment have been consistent with the original projections. At baseline, the mean age was 64 yrs; average lipid values were, in mg/dL: HDL-C 32; TG 161; LDL-C 111; total cholesterol 176. (For comparison, the mean baseline LDL-C in recent major secondary prevention trials was 189 in the Scandinavian Simvastatin Survival Study, 139 in the Cholesterol and Recurrent Events study, and 150 in the Long-Term Intervention with Pravastatin in Ischemic Disease study). The mean body mass index was 29±5. The prevalence of diabetes was 31% and of hypertension 57%. Aspirin and beta blockers were used by 82%, and 43% of patients, respectively.

Many heart disease patients have LDL-C cholesterol that is not high or even average. These patients often have low levels of the good cholesterol, HDL-C, as their primary lipid abnormality. Overall, approximately 25% of people with CHD, or about 3 million people in the United States, have this lipid profile (low LDL-C and low HDL-C).

VA-HIT was a major clinical trial to investigate whether lipid therapy benefits people with heart disease whose primary lipid abnormality is a low level of HDL-C. The VA-HIT study enrolled 2531 male veterans with heart disease who had low levels of LDL-C cholesterol and low levels of HDL-C cholesterol. The average LDL-C level was 111 mg/dL and HDL-C was 32 mg/dL. Patients were randomly assigned to receive either gemfibrozil or a placebo. Gemfibrozil, a fibric acid, significantly raised HDL-C and lowered triglycerides, another blood fat that may contribute to heart disease. Gemfibrozil did not lower LDL-C cholesterol.

The reduction in the incidence or severity of vascular disease was determined by comparing data from individuals undergoing the methods of the present invention to those who were not. Data regarding the incidence or occurrence of a vascular event including death were obtained from control patients having the same or similar disease states under the same or similar conditions as the individual undergoing the methods of the present invention. The control patient received a placebo, as opposed to the test patients who received the lipid modifying drug.

Data

2531 men with CHD enrolled in trial 1991-1993. The trial ended 7/31/98 with median follow-up of 5.1 years. Vital status of patients known is 99.2%. 71% remained on assigned therapy at end of trial which is about the average in a long trial.

TABLE 1
Percent change in Lipids

| | PLACEBO | GEMFIBROZIL |
|---|---|---|
| *total cholesterol | +2.1 | −2.9 |
| LDL-C | +4.0 | +3.6 |
| *HDL-C | +1.8 | +7.5 |
| *Triglycerides | +9.6 | −24.5 |

*Statically significant differences

TABLE 2
Primaiy Endpoints

| | | | RELATIVE RISK |
|---|---|---|---|
| | PLACEBO | GEMFIBROZIL | REDUCTION |
| CHD death/ | 22% | 17% | 22% |
| nonfatal MI | | | |

TABLE 3
Other Endpoints which were significantly benefitted by Gemfibrozil

| | INCIDENCE RATE | |
|---|---|---|
| | PLACEBO | GEMFIBROZIL |
| transient ischemia attack | 4.2% | 1.7% |
| (TIA) | | |
| carotid endarterectomy | 3.5% | 1.3% |
| total reported stroke | 7.0% | 5.0% |
| hospitalization for congestive | 13.3% | 10.6% |
| heart failure | | |

The benefit achieved in VA-HIT with gemfibrozil in reducing major CHD events in U.S.A. patients without an increased LDL-C was at least as great as with reductase inhibitors (e.g., statins) in reducing CHD endpoints in the patients with a high LDL-C.

The major findings of this seven year study were that:

Patients receiving gemfibrozil had a 22% reduction in the number of heart attacks and cardiac deaths, the primary study endpoint. This finding was highly statistically significant (p=0.0063).

Using the combined endpoint of heart attacks, cardiac deaths, and strokes patients receiving gemfibrozil had a 24% reduction in these major events (p=0.0011).

Gemfibrozil was effective in both diabetics and non-diabetics and in younger as well as older patients.

Gemfibrozil was not associated with any major adverse events. Major adverse events that were monitored included deaths from any cause and cancer.

VA-HIT data indicates that 18 patients would need to be treated with gemfibrozil over a time period (e.g., between 1 and 5 years) to prevent one heart attack, cardiac death, or stroke. VA-HIT study also provides direct clinical trial evidence of a beneficial effect of lipid therapy in CHD patients with low HDL-cholesterol and low LDL-cholesterol. This study demonstrates a clinical benefit from raising HDL-cholesterol and lowering triglycerides without lowering LDL-cholesterol.

### EXAMPLE 2

Fibric acid decreases incidence of vascular events in diabetic patients (e.g., events that cause the heart to have irregular rhythms such as ventricular fibrillation, ventricular tachycardia, asystole, and includes, for example, myocardial infarction, angina, and death).

Diabetes mellitus (DM) has long been recognized as a major risk factor for a vascular atherothrombotic event, most particularly coronary heart disease and cerebrovascular disease. The population of patients recruited for the Veterans Affairs HDL-C Intervention Trial (VA-HIT) with a low HDL-cholesterol (HDL-C) and a low LDL-cholesterol (LDL-C) had a high prevalence of DM. Although patients with DM were not singled out as a specific recruitment target (i.e., not a specific target for fibrate therapy in VA-HIT), patients with DM frequently have a low HDL-C and, thus, were clearly eligible for VA-HIT and treatment with the fibrate, genfibrozil.

In all, 25% of the 2531 patients enrolled in VA-HIT had a history of DM. An additional 6% of patients without a history of DM on entry into the study were found to have DM by glucose testing. Thus, 31% of VA-HIT patients had diabetes. In addition to these patients with overt DM, 16% of additional VA-HIT patients had a pre-diabetic state that is commonly referred to as “insulin resistance: and is detected in clinical practice by the presence of high fasting blood insulin levels. There is evidence that patients with high insulin levels (i.e., patients with “hyperinsulinemia” or “insulin resistance”) but without overt DM have as high a risk of coronary heart disease as is associated with DM itself. Consequently, 47% of patients in VA-HIT had either DM or hyperinsulinemia and in this large subgroup of patients, both coronary heart disease (CHD) events and stroke were higher than in the remainder of VA-HIT patients and the benefit of fibrate therapy was greater. Table 4 below shows these data:

TABLE 4
Effect of Gemfibrozil in the Reduction of Cardiovascular Risk

| | Five Year Event Rate | | | | Overall Risk |
|---|---|---|---|---|---|
| | Placebo Group | | Gemfibrozil Group | | Reduction with |
| VA-HIT | CHD | Stroke | CHD | Stroke | Gemfibrozil |
| Subgroups | % | % | % | % | % |
| Diabetes | 29.4 | 10.5 | 21.2 | 6.6 | 30.3 |
| Hyperinsul- | 27.7 | 8.1 | 20.9 | 6.2 | 24.2 |
| inemia* | | | | | |
| Hyperinsul- | 23.1 | 4.3 | 16.8 | 4.7 | 21.7 |
| inemia alone** | | | | | |
| All Others | 16.6 | 4.0 | 15.3 | 3.2 | 10.3 |

*Includes patients with hyperinsulinemia + diabetes
**Includes only patients with hyperinsulinemia

In further analysis, our data can show that in this large subgroup of patients with DM and hyperinsulinemia gemfibrozil was particularly effective in reducing CHD death (e.g., death followed by or as a result of CHD), the most meaningful of all categories commonly designated as a “CHD event”. Moreover, when patients with DM and isolated hyperinsulinemia were included together in an analysis, gemfibrozil was also shown to significantly reduce the 5-year incidence rate of all-cause mortality (Table 5).

TABLE 5
Effect of Gemfibrozil in the Reduction of CHD Death and All-Cause
Mortality in VA-HIT.

| | | RRR | (95% Confidence | |
|---|---|---|---|---|
| | N | % | Intervals) | P-value |
| CHD Death | | | | |
| Diabetes | 769 | 41 | (51, 9) | 0.016 |
| All-Cause Mortality | | | | |
| Diabetes | 769 | 25 | (44, −1) | 0.059 |
| Diabetes + | 1157 | 26 | (43, 4) | 0.021 |
| Hyperinsulinemia alone | | | | |

Abbreviation: N, number of patients; RRR, relative risk reduction (with gemfibrozil).

Until the discovery of the present invention, it has not been shown that a significant reduction in CHD deaths can be achieved in a diabetic population with drug therapy, including drugs that normalize blood sugar.

The teachings of all journal articles, abstracts, patents, patent applications, and patent publications cited herein are hereby incorporated by reference in their entirety.

1. A method of treating an individual who has diabetes and is at risk for a vascular disease, wherein the method comprises:
administering a fibric acid or derivative thereof in a carrier to the diabetic individual,
wherein the diabetic individual has an HDL-C level of less than about 40 mg/dL and an LDL-C level of less than about 130 mg/dL, a decrease in the incidence or severity of a vascular disease occurs and an increase in an HDL-C level in the diabetic individual occurs.

2. The method of claim 1, wherein the vascular disease is a thrombotic disorder, myocardial infarction, angina, a stroke, a transient ischemic attack, a thrombotic re-occlusion subsequent to a coronary intervention procedure, or a disorder in which at least one major coronary artery exhibits greater than 50% stenosis.

3. The method of claim 2, wherein the fibric acid or derivative thereof is gemfibrozil, fenofibrate, bezafibrate, ciprofibrate, clofibrate, or clinofibrate.

4. The method of claim 3, wherein the triglyceride level is reduced by at least about 25% after treatment.

5. The method of claim 1, wherein the HDL-C level is increased by at least about 5% after treatment.

6. A method of reducing the incidence or severity of a vascular disease in an individual who has diabetes, wherein the method comprises:
administering a fibric acid or derivative thereof in a carrier to the diabetic individual,
wherein the diabetic individual has an HDL-C level of less than about 40 mg/dL and an LDL-C level of less than about 130 mg/dL, a decrease in the incidence or severity of a vascular disease occurs and an increase in an HDL-C level in the individual occurs.

7. The method of claim 6, wherein the vascular disease is a thrombotic disorder, myocardial infarction, angina, a stroke, a transient ischemic attack, a thrombotic re-occlusion subsequent to a coronary intervention procedure, or a disorder in which at least one major coronary artery exhibits greater than 50% stenosis.

8. The method of claim 7, wherein the fibric acid or derivative thereof is gemfibrozil, ferofibrate, bezafibrate, ciprofibrate, clofibrate, or clinofibrate.

9. The method of claim 8, wherein the triglyceride level is reduced by at least about 25% after treatment.

10. The method of claim 6, wherein the HDL-C level is increased by at least about 5% after treatment.

11. A method of preventing vascular disease in an individual who has diabetes, wherein the method comprises:
administering a fibric acid or derivative thereof in a carrier to the individual having diabetes,
wherein the diabetic individual has an HDL-C level of less than about 40 mg/dL and an LDL-C level of less than about 130 mg/dL,; an increase in HDL-C occurs; and the onset of the vascular disease is delayed or prevented.

12. The method of claim 11, wherein the vascular event is a thrombotic disorder, myocardial infarction, angina, a stroke, a transient ischemic attack, a thrombotic re-occlusion subsequent to a coronary intervention procedure, or a disorder in which at least one major coronary artery exhibits greater than 50% stenosis.

13. The method of claim 12, wherein the fibric acid or derivative thereof is gemfibrozil, ferofibrate, bezafibrate, ciprofibrate, clofibrate, or clinofibrate.

14. The method of claim 13, wherein the triglyceride level is reduced by at least about 25% after treatment.

15. The method of claim 11, wherein the HDL-C level is increased by at least about 5% after treatment.

16. A method of reducing the incidence or severity of a cardiovascular or cerebrovascular event in an individual having a lipid profile of an HDL-C level of less than about 40 mg/dL prior to treatment, and an LDL-C level of less than about 130 mg/dL prior to treatment; wherein the individual is at risk for a cardiovascular or cerebrovascular event; wherein the method comprises:
administering an effective amount of a fibric acid or derivative thereof in a carrier to the individual;
wherein the fibric acid or derivative thereof is gemfibrozil, fenofibrate, bezafibrate, ciprofibrate, clofibrate or clinofibrate; the cardiovascular or cerebrovascular event is a thrombotic disorder, myocardial infarction, angina, a stroke, a transient ischemic attack, thrombotic re-occlusion subsequent to a coronary intervention procedure, or a disorder in which at least one major coronary artery exhibits greater than 50% stenosis; an increase of HDL-C occurs after treatment; and a reduction in the incidence or severity of a cardiovascular or a cerebrovascular occurs.

17. The method of claim 16, wherein the triglyceride level is reduced by at least about 25% after treatment.

18. The method of claim 16, wherein the HDL-C level is increased by at least about 5% after treatment.

19. A method for preventing the onset of a cardiovascular or cerebrovascular event in an individual having a lipid profile of an HDL-C level of less than about 40 mg/dL prior to treatment, and an LDL-C level of less than about 130 mg/dL prior to treatment; wherein the individual is at risk for a cardiovascular or cerebrovascular event; wherein the method comprises:
administering an effective amount of a fibric acid or derivative thereof in a carrier to the individual;
wherein the fibric acid or derivative thereof is gemfibrozil, fenofibrate, bezafibrate, ciprofibrate, clofibrate or clinofibrate; the cardiovascular or cerebrovascular event is a thrombotic disorder, myocardial infarction, angina, a stroke, a transient ischemic attack, thrombotic re-occlusion subsequent to a coronary intervention procedure, or a disorder in which at least one major coronary artery exhibits greater than 50% stenosis; an increase of HDL-C occurs after treatment; and the onset of the cardiovascular or cerebrovascular event is delayed or prevented.

20. The method of claim 19, wherein the triglyceride level is reduced by at least about 25% after treatment.

21. The method of claim 19, wherein the HDL-C level is increased by at least about 5% after treatment.
